(19) United States
(12) Patent Application Publication
(10) Pub. No.: US 2020/0325116 A1
(43) Pub. Date: Oct. 15, 2020

(54) SOLID FORMS OF A PLASMA KALLIKREIN INHIBITOR AND SALTS THEREOF

(71) Applicant: KALVISTA PHARMACEUTICALS LIMITED, Wiltshire (GB)

(72) Inventors: David Malcolm CROWE, Berkshire (GB); David Michael EVANS, Wiltshire (GB)

(21) Appl. No.: 16/768,202

(22) PCT Filed: Nov. 29, 2018
(86) PCT No.: PCT/GB2018/053464

§ 371 Date: May 29, 2020

Related U.S. Application Data
(60) Provisional application No. 62/592,160, filed on Nov. 29, 2017.

(30) Foreign Application Priority Data
Nov. 29, 2017 (GB) 1719882.1

(51) Int. Cl. C07D 401/14; A61K 9/00

(52) U.S. Cl. CPC C07D 401/14; A61K 9/0019; A61K 9/0048

(57) ABSTRACT

The present invention relates to new solid forms of a plasma kallikrein inhibitor, a pharmaceutical composition containing them and their use in therapy. Also provided are processes for preparing the solid forms of the present invention.

The present invention relates to new solid forms of a plasma kallikrein inhibitor, a pharmaceutical composition containing them and their use in therapy. Also provided are processes for preparing the solid forms of the present invention.

### BACKGROUND TO THE INVENTION

Inhibitors of plasma kallikrein have a number of therapeutic applications, particularly in the treatment of retinal vascular permeability associated with diabetic retinopathy, diabetic macular edema and hereditary angioedema.

Plasma kallikrein is a trypsin-like serine protease that can liberate kinins from kininogens (see K. D. Bhoola et al., “Kallikrein-Kinin Cascade”, Encyclopedia of Respiratory Medicine, p 483-493; J. W. Bryant et al., “Human plasma kallikrein-kinin system: physiological and biochemical parameters” Cardiovascular and haematological agents in medicinal chemistry, 7, p 234-250, 2009; K. D. Bhoola et al., Pharmacological Rev., 1992, 44, 1; and D. J. Campbell, “Towards understanding the kallikrein-kinin system: insights from the measurement of kinin peptides”, Brazilian Journal of Medical and Biological Research 2000, 33, 665-677). It is an essential member of the intrinsic blood coagulation cascade although its role in this cascade does not involve the release of bradykinin or enzymatic cleavage. Plasma prekallikrein is encoded by a single gene and synthesized in the liver. It is secreted by hepatocytes as an inactive plasma prekallikrein that circulates in plasma as a heterodimer complex bound to high molecular weight kininogen which is activated to give the active plasma kallikrein. Kinins are potent mediators of inflammation that act through G protein-coupled receptors and antagonists of kinins (such as bradykinin antagonists) have previously been investigated as potential therapeutic agents for the treatment of a number of disorders (F. Marceau and D. Regoli, Nature Rev., Drug Discovery, 2004, 3, 845-852).

Plasma kallikrein is thought to play a role in a number of inflammatory disorders. The major inhibitor of plasma kallikrein is the serpin C1 esterase inhibitor. Patients who present with a genetic deficiency in C1 esterase inhibitor suffer from hereditary angioedema (HAE) which results in intermittent swelling of face, hands, throat, gastro-intestinal tract and genitals. Blisters formed during acute episodes contain high levels of plasma kallikrein which cleaves high molecular weight kininogen liberating bradykinin leading to increased vascular permeability. Treatment with a large protein plasma kallikrein inhibitor has been shown to effectively treat HAE by preventing the release of bradykinin which causes increased vascular permeability (A. Lehmann “Ecallantide (DX-88), a plasma kallikrein inhibitor for the treatment of hereditary angioedema and the prevention of blood loss in on-pump cardiothoracic surgery” Expert Opin. Biol. Ther. 8, p 1187-99).

The plasma kallikrein-kinin system is abnormally abundant in patients with advanced diabetic macular edema. It has been recently published that plasma kallikrein contributes to retinal vascular dysfunctions in diabetic rats (A. Clermont et al. “Plasma kallikrein mediates retinal vascular dysfunction and induces retinal thickening in diabetic rats” Diabetes, 2011, 60, p 1590-98). Furthermore, administration of the plasma kallikrein inhibitor ASP-440 ameliorated both retinal vascular permeability and retinal blood flow abnormalities in diabetic rats. Therefore a plasma kallikrein inhibitor should have utility as a treatment to reduce retinal vascular permeability associated with diabetic retinopathy and diabetic macular edema.

Plasma kallikrein also plays a role in blood coagulation. The intrinsic coagulation cascade may be activated by factor XII (FXII). Once FXII is activated (to FXIIa), FXIIa triggers fibrin formation through the activation of factor XI (FXI) thus resulting in blood coagulation. Plasma kallikrein is a key component in the intrinsic coagulation cascade because it activates FXII to FXIIa, thus resulting in the activation of the intrinsic coagulation pathway. Furthermore, FXIIa also activates further plasma prekallikrein resulting in plasma kallikrein. This results in positive feedback amplification of the plasma kallikrein system and the intrinsic coagulation pathway (Tanaka et al. (Thrombosis Research 2004, 113, 333-339); Bird et al. (Thrombosis and Haemostasis, 2012, 107, 1141-50).

Contact of FXII in the blood with negatively charged surfaces (such as the surfaces of external pipes or the membrane of the oxygenator that the blood passes during cardiopulmonary bypass surgery) induces a conformational change in zymogen FXII resulting in a small amount of active FXII (FXIIa). The formation of FXIIa triggers the formation of plasma kallikrein resulting in blood coagulation, as described above. Activation of FXII to FXIIa can also occur in the body by contact with negatively charged surfaces on various sources (e.g. bacteria during sepsis, RNA from degrading cells), thus resulting in disseminated intravascular coagulation (Tanaka et al. (Thrombosis Research 2004, 113, 333-339)).

Therefore, inhibition of plasma kallikrein would inhibit the blood coagulation cascade described above, and so would be useful in the treatment of disseminated intravascular coagulation and blood coagulation during cardiopulmonary bypass surgery where blood coagulation is not desired. For example, Katsuura et al. (Thrombosis Research, 1996, 82, 361-368) showed that administration of a plasma kallikrein inhibitor, PKSI-527, for LPS-induced disseminated intravascular coagulation significantly suppressed the decrease in platelet count and fibrinogen level as well as the increase in FDP level which usually occur in disseminated intravascular coagulation. Bird et al. (Thrombosis and Haemostasis, 2012, 107, 1141-50) showed that clotting time increased, and thrombosis was significantly reduced in plasma kallikrein-deficient mice. Revenko et al. (Blood, 2011, 118, 5302-5311) showed that the reduction of plasma prekallikrein levels in mice using antisense oligonucleotide treatment resulted in antithrombotic effects. Tanaka et al. (Thrombosis Research 2004, 113, 333-339) showed that contacting blood with DX-88 (a plasma kallikrein inhibitor) resulted in an increase in activated clotting time (ACT). Lehmann et al. (Expert Opin. Biol. Ther. 2008, 1187-99) showed that Ecallantide (a plasma kallikrein inhibitor) was found to delay contact activated induced coagulation. Lehmann et al. conclude that Ecallantide “had in vitro anticoagulant effects as it inhibited the intrinsic pathway of coagulation by inhibiting plasma kallikrein”.

Plasma kallikrein also plays a role in the inhibition of platelet activation, and therefore the cessation of bleeding. Platelet activation is one of the earliest steps in hemostasis, which leads to platelet plug formation and the rapid cessation of bleeding following damage to blood vessels. At the site of vascular injury, the interaction between the exposed collagen and platelets is critical for the retention and activation of platelets, and the subsequent cessation of bleeding.

Once activated, plasma kallikrein binds to collagen and thereby interferes with collagen-mediated activation of platelets mediated by GPVI receptors (Liu et al. (Nat Med., 2011, 17, 206-210)). As discussed above, plasma kallikrein inhibitors reduce plasma prekallikrein activation by inhibiting plasma kallikrein-mediated activation of factor XII and thereby reducing the positive feedback amplification of the kallikrein system by the contact activation system.

Therefore, inhibition of plasma kallikrein reduces the binding of plasma kallikrein to collagen, thus reducing the interference of plasma kallikrein in the cessation of bleeding. Therefore plasma kallikrein inhibitors would be useful in the treatment of treating cerebral haemorrhage and bleeding from post operative surgery. For example, Liu et al. (Nat Med., 2011, 17, 206-210) demonstrated that systemic administration of a small molecule PK inhibitor, ASP-440, reduced hematoma expansion in rats. Cerebral hematoma may occur following intracerebral haemorrhage and is caused by bleeding from blood vessels into the surrounding brain tissue as a result of vascular injury. Bleeding in the cerebral haemorrhage model reported by Liu et al. was induced by surgical intervention involving an incision in the brain parenchyma that damaged blood vessels. These data demonstrate that plasma kallikrein inhibition reduced bleeding and hematoma volume from post operative surgery. Björkqvist et al. (Thrombosis and Haemostasis, 2013, 110, 399-407) demonstrated that aprotinin (a protein that inhibits serine proteases including plasma kallikrein) may be used to decrease postoperative bleeding.

Other complications of diabetes such as cerebral haemorrhage, nephropathy, cardiomyopathy and neuropathy, all of which have associations with plasma kallikrein may also be considered as targets for a plasma kallikrein inhibitor.

Synthetic and small molecule plasma kallikrein inhibitors have been described previously, for example by Garrett et al. (“Peptide aldehyde . . . ” J. Peptide Res. 52, p 62-71 (1998)), T. Griesbacher et al. (“Involvement of tissue kallikrein but not plasma kallikrein in the development of symptoms mediated by endogenous kinins in acute pancreatitis in rats” British Journal of Pharmacology 137, p 692-700 (2002)), Evans (“Selective dipeptide inhibitors of kallikrein” WO03/076458), Szelke et al. (“Kininogenase inhibitors” WO92/04371), D. M. Evans et al. (Immunolpharmacology, 32, p 115-116 (1996)), Szelke et al. (“Kininogen inhibitors” WO95/07921), Antonsson et al. (“New peptides derivatives” WO94/29335), J. Corte et al. (“Six membered heterocycles useful as serine protease inhibitors” WO2005/123680), J. Stürzbecher et al. (Brazilian J. Med. Biol. Res 27, p 1929-34 (1994)), Kettner et al. (U.S. Pat. No. 5,187,157), N. Teno et al. (Chem. Pharm. Bull. 41, p 1079-1090 (1993)), W. B. Young et al. (“Small molecule inhibitors of plasma kallikrein” Bioorg. Med. Chem. Letts. 16, p 2034-2036 (2006)), Okada et al. (“Development of potent and selective plasmin and plasma kallikrein inhibitors and studies on the structure-activity relationship” Chem. Pharm. Bull. 48, p 1964-72 (2000)), Steinmetzer et al. (“Trypsin-like serine protease inhibitors and their preparation and use” WO08/049595), Zhang et al. (“Discovery of highly potent small molecule kallikrein inhibitors” Medicinal Chemistry 2, p 545-553 (2006)), Sinha et al. (“Inhibitors of plasma kallikrein” WO08/016883), Shigenaga et al. (“Plasma Kallikrein Inhibitors” WO2011/118672), and Kolte et al. (“Biochemical characterization of a novel high-affinity and specific kallikrein inhibitor”, British Journal of Pharmacology (2011), 162(7), 1639-1649). Also, Steinmetzer et al. (“Serine protease inhibitors” WO2012/004678) describes cyclized peptide analogs which are inhibitors of human plasmin and plasma kallikrein.

To date, the only selective plasma kallikrein inhibitor approved for medical use is Ecallantide. Ecallantide is formulated as a solution for injection. It is a large protein plasma kallikrein inhibitor that presents a risk of anaphylactic reactions. Other plasma kallikrein inhibitors known in the art are generally small molecules, some of which include highly polar and ionisable functional groups, such as guanidines or amidines. Recently, plasma kallikrein inhibitors that do not feature guanidine or amidine functionalities have been reported. For example Brandi et al. (“N-((6-amino-pyridin-3-yl)methyl)-heteroaryl-carboxamides as inhibitors of plasma kallikrein” WO2012/017020), Evans et al. (“Benzylamine derivatives as inhibitors of plasma kallikrein” WO2013/005045), Allan et al. (“Benzylamine derivatives” WO2014/108679), Davie et al. (“Heterocyclic derivates” WO2014/188211), and Davie et al. (“N-((het)arylmethyl)-heteroaryl-carboxamides compounds as plasma kallikrein inhibitors” WO2016/083820).

In the manufacture of pharmaceutical formulations, it is important that the active compound be in a form in which it can be conveniently handled and processed in order to obtain a commercially viable manufacturing process. Accordingly, the chemical stability and the physical stability of the active compound are important factors. The active compound, and formulations containing it, must be capable of being effectively stored over appreciable periods of time, without exhibiting any significant change in the physico-chemical characteristics (e.g. chemical composition, density, hygroscopicity and solubility) of the active compound.

It is known that manufacturing a particular solid-state form of a pharmaceutical ingredient can affect many aspects of its solid state properties and offer advantages in aspects of solubility, dissolution rate, chemical stability, mechanical properties, technical feasibility, processability, pharmacokinetics and bioavailability. Some of these are described in “Handbook of Pharmaceutical Salts; Properties, Selection and Use”, P. Heinrich Stahl, Camille G. Wermuth (Eds.) (Verlag Helvetica Chimica Acta, Zurich). Methods of manufacturing solid-state forms are also described in “Practical Process Research and Development”, Neal G. Anderson (Academic Press, San Diego) and “Polymorphism: In the Pharmaceutical Industry”, Rolf Hilfiker (Ed) (Wiley VCH). Polymorphism in pharmaceutical crystals is described in Byrn (Byrn, S. R., Pfeiffer, R. R., Stowell, J. G., “Solid-State Chemistry of Drugs”, SSCI Inc., West Lafayette, Ind., 1999), Brittain, H. G., “Polymorphism in Pharmaceutical Solids”, Marcel Dekker, Inc., New York, Basel, 1999) or Bernstein (Bernstein, J., “Polymorphism in Molecular Crystals”, Oxford University Press, 2002).

The applicant has developed a novel series of compounds that are inhibitors of plasma kallikrein, which are disclosed in PCT/GB2017/051546. These compounds demonstrate good selectivity for plasma kallikrein and are potentially useful in the treatment of diabetic retinopathy, macular edema and hereditary angioedema. One such compound is N-[(3-fluoro-4-methoxypyridin-2-yl)methyl]-1-({4-[(2-oxopyridin-1-yl)methyl]phenyl}methyl)-3-(trifluoromethyl)pyrazole-4-carboxamide (Example 6 of PCT/GB2017/051546).

The name N-[(3-fluoro-4-methoxypyridin-2-yl)methyl]-1-({4-[(2-oxopyridin-1-yl)methyl]phenyl}methyl)-3-(trifluoromethyl)pyrazole-4-carboxamide denotes the structure depicted in Formula A.

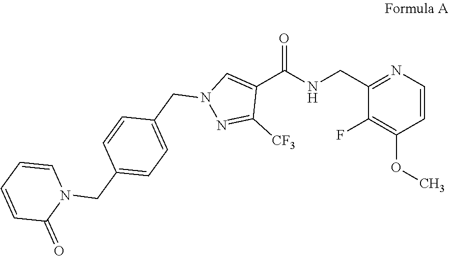 [SMILES: COc1ccnc(CNC(=O)c2cn(Cc3ccc(Cn4ccccc4=O)cc3)nc2C(F)(F)F)c1F]

The applicant has also developed novel solid forms of the related compound N-[(3-fluoro-4-methoxypyridin-2-yl)methyl]-3-(methoxymethyl)-1-({4-[(2-oxopyridin-1-yl)methyl]phenyl}methyl)pyrazole-4-carboxamide and salts thereof which are disclosed in PCT/GB2017/051579. These solid forms have advantageous physico-chemical properties that render them suitable for development.

### DESCRIPTION OF THE INVENTION

The applicant has now developed a novel solid form of the compound of Formula A, which is herein referred to as ‘Form 1’. Form 1 has advantageous physico-chemical properties that render it suitable for development, in particular, its preparation by crystallisation is simple and scalable. An advantage of crystalline solid forms is that they are more easily processable. That is, their preparation by crystallisation is a common and easily scalable procedure to remove undesirable impurities.

Furthermore, the compound of Formula A has been found to demonstrate surprisingly good pharmacokinetic properties, in particular, in vitro permeability.

Thus, in accordance with an aspect of the present invention, there is provided a solid form of the compound of Formula A. In the present application this solid form is referred to as ‘Form 1’.

The applicant has also developed a novel solid form of the hydrochloride salt of the compound of Formula A. The novel solid form has advantageous physico-chemical properties that render it suitable for development, in particular, its preparation by crystallisation is simple and scalable.

The present invention provides a solid form of the hydrochloride salt of the compound of Formula A which is herein referred to as ‘Form 2’.

The applicant has also developed a novel solid form of the sulfate salt of the compound of Formula A. The novel solid form has advantageous physico-chemical properties that render it suitable for development, in particular, its preparation by crystallisation is simple and scalable.

The present invention provides a solid form of the sulfate salt of the compound of Formula A which is herein referred to as ‘Form 3’.

The term “sulfate” as used herein when referring to a salt of the compound of Formula A is intended to encompass both a mono-sulfate salt and a hemi-sulfate salt. In one embodiment, Form 3 of the compound of Formula A is a mono-sulfate salt. In an alternative embodiment, Form 3 of the compound of Formula A is a hemi-sulfate salt.

The term “solid forms” described herein includes crystalline forms. Optionally, the solid forms of the invention are crystalline forms.

In the present specification, X-ray powder diffraction peaks (expressed in degrees 2θ) are measured using Cu Kα radiation.

The present invention provides a solid form (Form 1) of the compound of Formula A, which exhibits at least the following characteristic X-ray powder diffraction peaks (Cu Kα radiation, expressed in degrees 2θ) at approximately:

(1) 6.7, 9.5, 11.0, 13.3, and 17.3; or

(2) 6.7, 8.2, 9.5, 11.0, 13.3, 15.6, and 17.3; or

(3) 6.7, 8.2, 9.5, 11.0, 13.3, 14.5, 15.6, 17.3, and 20.5.

The term “approximately” means in this context that there is an uncertainty in the measurements of the degrees 2θ of ±0.3 (expressed in degrees 2θ), preferably ±0.2 (expressed in degrees 2θ).

The present invention also provides a solid form (Form 1) of the compound of Formula A, having an X ray powder diffraction pattern comprising characteristic peaks (expressed in degrees 2θ) at approximately 6.7, 8.2, 9.5, 11.0, 13.3, 13.7, 14.5, 15.6, 17.3, 19.1, and 20.5.

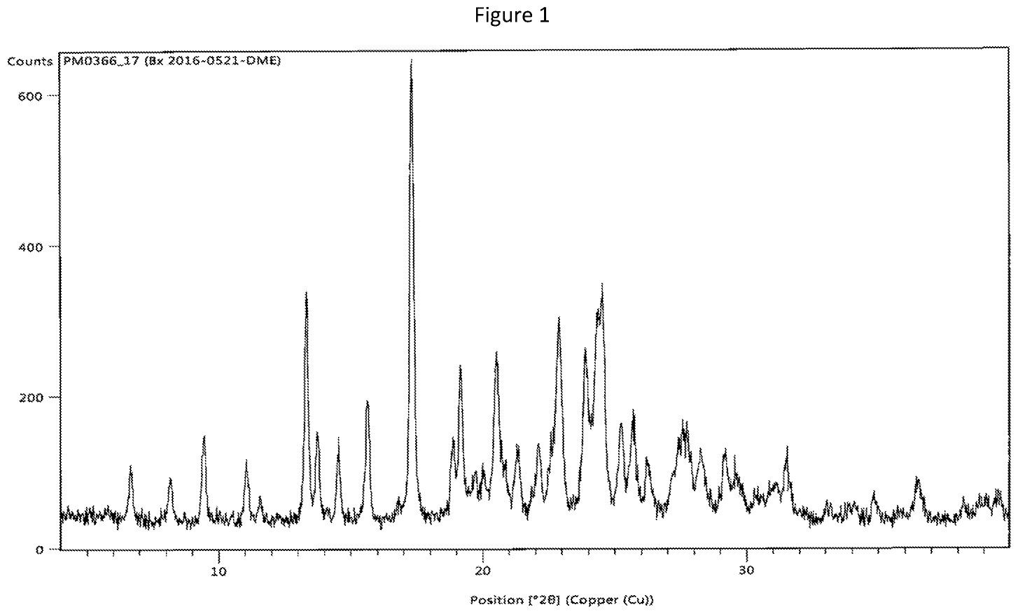

The present invention also provides a solid form (Form 1) of the compound of Formula A having an X-ray powder diffraction pattern substantially the same as that shown in FIG. 1.

The X-ray powder diffraction pattern of a solid form may be described herein as “substantially” the same as that depicted in a Figure. It will be appreciated that the peaks in X-ray powder diffraction patterns may be slightly shifted in their positions and relative intensities due to various factors known to the skilled person. For example, shifts in peak positions or the relative intensities of the peaks of a pattern can occur because of the equipment used, method of sample preparation, preferred packing and orientations, the radiation source, and method and length of data collection. However, the skilled person will be able to compare the X-ray powder diffraction patterns shown in the figures herein with those of an unknown solid form to confirm the identity of the solid form.

The present invention provides a solid form (Form 2) of the hydrochloride salt of the compound of Formula A, which exhibits at least the following characteristic X-ray powder diffraction peaks (Cu Kα radiation, expressed in degrees 2θ) at approximately:

(1) 7.3, 8.6, 11.6, 14.3, and 16.2; or

(2) 7.3, 8.6, 11.6, 13.5, 14.3, 16.2, and 18.3; or

(3) 7.3, 8.6, 11.2, 11.6, 13.5, 14.3, 14.7, 16.2, and 18.3.

The present invention also provides a solid form (Form 2) of the hydrochloride salt of the compound of Formula A, having an X ray powder diffraction pattern comprising characteristic peaks (expressed in degrees 2θ) at approximately 7.3, 7.8, 8.6, 11.2, 11.6, 13.5, 14.3, 14.7, 16.2, 17.2, 17.7, and 18.3.

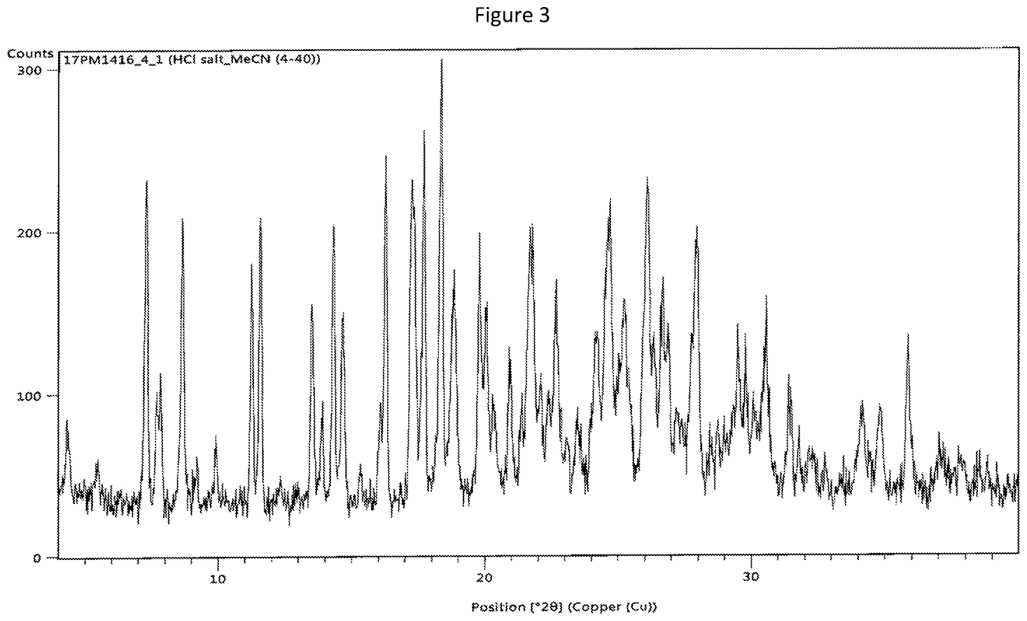

The present invention also provides a solid form (Form 2) of the hydrochloride salt of the compound of Formula A having an X-ray powder diffraction pattern substantially the same as that shown in FIG. 3.

The present invention provides a solid form (Form 3) of the sulfate salt of the compound of Formula A, which exhibits at least the following characteristic X-ray powder diffraction peaks (Cu Kα radiation, expressed in degrees 2θ) at approximately:

(1) 4.7, 6.4, 9.1, 15.1, and 16.4; or

(2) 4.7, 6.4, 9.1, 15.1, 16.4, 18.4, and 19.5.

The present invention also provides a solid form (Form 3) of the sulfate salt of the compound of Formula A, having an X ray powder diffraction pattern comprising characteristic peaks (expressed in degrees 2θ) at approximately 4.7, 6.4, 9.1, 12.8, 15.1, 16.4, 18.4, 18.8, and 19.5.

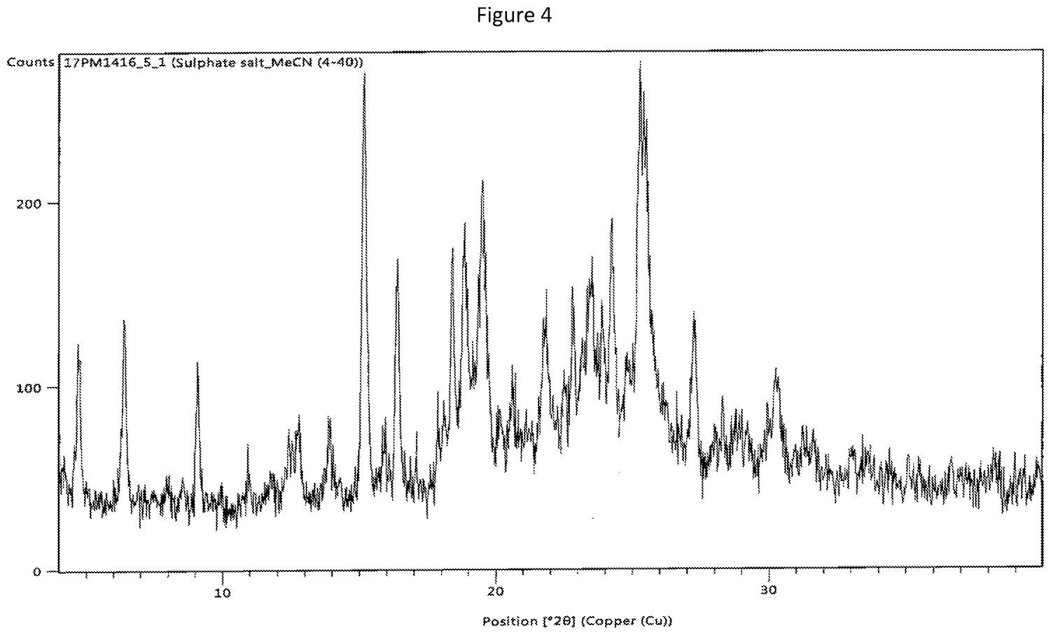

The present invention also provides a solid form (Form 3) of the sulfate salt of the compound of Formula A having an X-ray powder diffraction pattern substantially the same as that shown in FIG. 4.

The skilled person is familiar with techniques for measuring XRPD patterns. In particular, the X-ray powder diffraction pattern of the sample of compound may be recorded using a Philips X-Pert MPD diffractometer with the following experimental conditions:

Scan parameters:

| | |
|---|---|
| Scan Axis: | Gonio |
| Start Position [°2θ]: | 4.0084 |
| End Position [°2θ]: | 39.9804 |
| Step Size [°2θ]: | 0.0170 |
| Scan Step Time [s]: | 10.1600 |
| Scan Type: | Continuous |
| PSD Mode: | Scanning |
| PSD Length [°2θ]: | 2.12 |
| Offset [°2θ]: | 0.0000 |
| Divergence Slit Type: | Automatic |
| Irradiated Length [mm] | 10.00 |
| Specimen Length [mm]: | 10.00 |
| Measurement Temperature [° C.]: | 25.00 |
| Anode Material: | Cu |
| K-Alpha1 [Å]: | 1.54060 |
| K-Alpha2 [Å]: | 1.54443 |
| K-A2/K-A1 Ratio: | 0.50000 |
| Generator Settings: | 40 mA, 40 kV |
| Diffractometer Type: | 0000000011038600 |
| Diffractometer Number: | 0 |
| Goniometer Radius [mm]: | 240.00 |
| Dist. Focus-Diverg. Slit [mm]: | 100.00 |
| Incident Beam Monochromator: | No |
| Spinning: | Yes |

Sample: Approximately 5 mg of sample under analysis gently compressed on the XRPD zero back ground single obliquely cut silica sample holder.

The present invention provides a solid form (Form 1) of the compound of Formula A, which exhibits an endothermic peak in its STA thermograph at 164±3° C., preferably 164±2° C., more preferably 164±1° C.

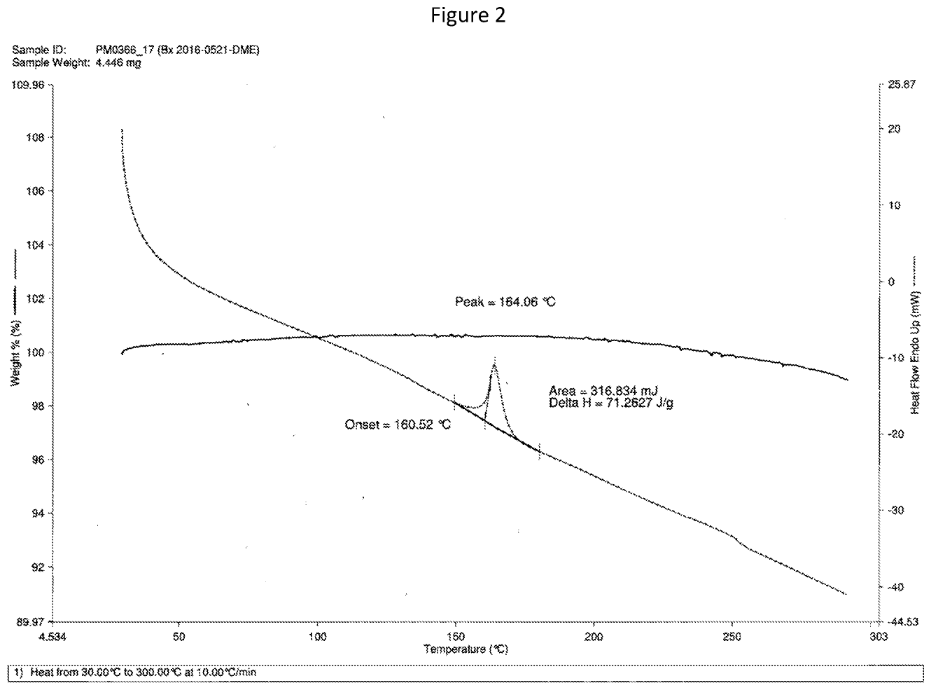

The present invention provides a solid form (Form 1) of the compound of Formula A, having an STA thermograph substantially the same as that shown in FIG. 2.

The skilled person is familiar with techniques for measuring STA thermographs. In particular, the STA thermograph of the sample of compound may be recorded by

(a) weighing approximately 5 mg of sample into a ceramic crucible;

(b) loading the sample into the chamber of Perkin-Elmer STA 600 TGA/DTA analyzer at ambient temperature;

(c) heating the sample from 25° C. to 300° C. at a rate of 10° C./min, and monitoring the change in weight of the sample as well as DTA signal while using a 20 cm³/min nitrogen purge.

The present invention provides a solid form (Form 1) of the compound of Formula A having an X-ray powder diffraction pattern as described above, and an STA thermograph as described above.

The solid form of the present invention can exist in both unsolvated and solvated forms. The term ‘solvate’ is used herein to describe a molecular complex comprising the compound of the invention and an amount of one or more pharmaceutically acceptable solvents, for example, ethanol. The term ‘hydrate’ is employed when the solvent is water.

The present invention encompasses solvates (e.g. hydrates) of the solid forms of the compound of Formula A and salts thereof described herein.

In an aspect of the invention, Form 1 of the compound of Formula A is not a solvate or a hydrate.

In an aspect of the invention, Form 2 of the compound of Formula A is a dihydrate.

In an aspect of the invention, Form 3 of the compound of Formula A is a monohydrate.

A reference to a particular compound also includes all isotopic variants.

The present invention also encompasses a process for the preparation of Form 1 of the present invention, said process comprising the crystallisation of said solid form from a solution of the compound of Formula A in a solvent or a mixture of solvents. The solvent or mixture of solvents may comprise isopropanol (IPA). Preferably the solvent is isopropanol. After adding the compound of Formula A to a solvent or a mixture of solvents (e.g. isopropanol), the combined mixture (compound plus solvent(s)) may be heated to a temperature of approximately 60-85° C. Alternatively, the combined mixture may be heated to a temperature of approximately 70-85° C. Alternatively, the combined mixture may be heated to a temperature of approximately 80-85° C. Alternatively, the combined mixture may be heated to a temperature of approximately 80, 81, 82, 83, 84 or 85° C. Alternatively, the combined mixture may be heated to a temperature of approximately 82° C. Alternatively, the combined mixture may be heated to reflux. Following heating, the combined mixture may be cooled. Alternatively, the combined mixture may be cooled to a temperature of approximately 0-40° C. Alternatively, the combined mixture may be cooled to a temperature of approximately 10-30° C. Alternatively, the combined mixture may be cooled to room temperature. Alternatively, the combined mixture may be cooled to approximately 0° C.

The present invention also encompasses a process for the preparation of a solid form of the compound of Formula A, said process comprising the crystallisation of said solid form from a solution of the compound of Formula A in isopropanol (IPA). After adding the compound of Formula A to the isopropanol, the combined mixture (compound plus isopropanol) may be heated to a temperature of approximately 60-85° C. Alternatively, the combined mixture may be heated to a temperature of approximately 70-85° C. Alternatively, the combined mixture may be heated to a temperature of approximately 80-85° C. Alternatively, the combined mixture may be heated to a temperature of approximately 80, 81, 82, 83, 84 or 85° C. Alternatively, the combined mixture may be heated to a temperature of approximately 82° C. Alternatively, the combined mixture may be heated to reflux. Following heating, the combined mixture may be cooled. Alternatively, the combined mixture may be cooled to a temperature of approximately 0-40° C. Alternatively, the combined mixture may be cooled to a temperature of approximately 10-30° C. Alternatively, the combined mixture may be cooled to room temperature. Alternatively, the combined mixture may be cooled to approximately 0° C.

The present invention also encompasses a solid form of the compound of Formula A obtainable by a process comprising the crystallisation of said solid form from a solution of the compound of Formula A in isopropanol (IPA). After adding the compound of Formula A to the isopropanol, the combined mixture (compound plus isopropanol) may be heated to a temperature of approximately 60-85° C. Alternatively, the combined mixture may be heated to a temperature of approximately 70-85° C. Alternatively, the combined mixture may be heated to a temperature of approximately 80-85° C. Alternatively, the combined mixture may be heated to a temperature of approximately 80, 81, 82, 83, 84 or 85° C. Alternatively, the combined mixture may be heated to a temperature of approximately 82° C. Alternatively, the combined mixture may be heated to reflux. Following heating, the combined mixture may be cooled. Alternatively, the combined mixture may be cooled to a temperature of approximately 0-40° C. Alternatively, the combined mixture may be cooled to a temperature of approximately 10-30° C. Alternatively, the combined mixture may be cooled to room temperature. Alternatively, the combined mixture may be cooled to approximately 0° C.

The present invention also encompasses a process for the preparation of Form 2 of the present invention, said process comprising the crystallisation of said solid form from a solution of the hydrochloride salt of the compound of Formula A in a solvent or a mixture of solvents. Optionally, said solution of the hydrochloride salt of the compound of Formula A may be formed by adding hydrochloric acid to a solution or suspension of the compound of Formula A in a solvent or a mixture of solvents.

The present invention also encompasses a process for the preparation of Form 3 of the present invention, said process comprising the crystallisation of said solid form from a solution of the sulfate salt of the compound of Formula A in a solvent or a mixture of solvents. Optionally, said solution of the sulfate salt of the compound of Formula A may be formed by adding sulfuric acid to a solution or suspension of the compound of Formula A in a solvent or a mixture of solvents.

The processes of the present invention may also comprise the addition of crystalline seeds of the solid form of the invention.

In an aspect, the present invention provides the solid form of the invention when manufactured by a process according to the invention.

As previously mentioned, the solid form of the present invention has a number of therapeutic applications, particularly in the treatment of diseases or conditions mediated by plasma kallikrein.

Accordingly, the present invention provides a solid form of the compound of Formula A and salts thereof, as hereinbefore defined, for use in therapy. In a preferred embodiment, the solid form is Form 1.

The present invention also provides for the use of a solid form of the compound of Formula A and salts thereof, as hereinbefore defined, in the manufacture of a medicament for the treatment of a disease or condition mediated by plasma kallikrein. In a preferred embodiment, the solid form is Form 1.

The present invention also provides a solid form of the compound of Formula A and salts thereof, as hereinbefore defined, for use in a method of treatment of a disease or condition mediated by plasma kallikrein. In a preferred embodiment, the solid form is Form 1.

The present invention also provides a method of treatment of a disease or condition mediated by plasma kallikrein, said method comprising administering to a mammal in need of such treatment a therapeutically effective amount of a solid form of the compound of Formula A and salts thereof, as hereinbefore defined. In a preferred embodiment, the solid form is Form 1.

In an aspect, the disease or condition mediated by plasma kallikrein is selected from impaired visual acuity, diabetic retinopathy, retinal vascular permeability associated with diabetic retinopathy, diabetic macular edema, hereditary angioedema, retinal vein occlusion, diabetes, pancreatitis, cerebral haemorrhage, nephropathy, cardiomyopathy, neuropathy, inflammatory bowel disease, arthritis, inflammation, septic shock, hypotension, cancer, adult respiratory distress syndrome, disseminated intravascular coagulation, blood coagulation during cardiopulmonary bypass surgery, and bleeding from post-operative surgery. In a preferred embodiment, the disease or condition mediated by plasma kallikrein is diabetic macular edema. In another preferred embodiment, the disease or condition mediated by plasma kallikrein is hereditary angioedema.

Alternatively, the disease or condition mediated by plasma kallikrein may be selected from retinal vascular permeability associated with diabetic retinopathy, diabetic macular edema and hereditary angioedema. Alternatively, the disease or condition mediated by plasma kallikrein may be retinal vascular permeability associated with diabetic retinopathy or diabetic macular edema. The solid forms of the compound of Formula A and salts thereof may be administered in a form suitable for injection into the ocular region of a patient, in particular, in a form suitable for intra-vitreal injection.

In the context of the present invention, references herein to “treatment” include references to curative, palliative and prophylactic treatment, unless there are specific indications to the contrary. The terms “therapy”, “therapeutic” and “therapeutically” should be construed in the same way.

The solid form of the present invention may be administered alone or in combination with one or more other drugs. Generally, it will be administered as a formulation in association with one or more pharmaceutically acceptable excipients. The term “excipient” is used herein to describe any ingredient other than the compound(s) of the invention which may impart either a functional (i.e., drug release rate controlling) and/or a non-functional (i.e., processing aid or diluent) characteristic to the formulations. The choice of excipient will to a large extent depend on factors such as the particular mode of administration, the effect of the excipient on solubility and stability, and the nature of the dosage form.

In another aspect, the compounds of the present invention may be administered in combination with laser treatment of the retina. The combination of laser therapy with intravitreal injection of an inhibitor of VEGF for the treatment of diabetic macular edema is known (Elman M, Aiello L, Beck R, et al. “Randomized trial evaluating ranibizumab plus prompt or deferred laser or triamcinolone plus prompt laser for diabetic macular edema”. Ophthalmology. 27 Apr. 2010).

Pharmaceutical compositions suitable for the delivery of the solid form of the present invention and methods for their preparation will be readily apparent to those skilled in the art. Such compositions and methods for their preparation may be found, for example, in Remington's Pharmaceutical Sciences, 19th Edition (Mack Publishing Company, 1995).

For administration to human patients, the total daily dose of the solid form of the invention is typically in the range 0.1 mg and 10,000 mg, or between 1 mg and 5000 mg, or between 10 mg and 1000 mg depending, of course, on the mode of administration. If administered by intra-vitreal injection a lower dose of between 0.0001 mg (0.1 μg) and 0.2 mg (200 μg) per eye is envisaged, or between 0.0005 mg (0.5 μg) and 0.05 mg (50 μg) per eye.

The total daily dose may be administered in single or divided doses and may, at the physician's discretion, fall outside of the typical range given herein. These dosages are based on an average human subject having a weight of about 60 kg to 70 kg. The physician will readily be able to determine doses for subjects whose weight falls outside this range, such as infants and the elderly.

Accordingly, the present invention provides a pharmaceutical composition comprising a solid form of the compound of Formula A, as hereinbefore defined, and a pharmaceutically acceptable carrier, diluent and/or excipient. In a preferred embodiment, the solid form is Form 1. It will be appreciated that the reference to solid forms of the compound of Formula A as hereinbefore defined includes both the free base and the salts thereof which have hereinbefore been described.

The pharmaceutical compositions may be administered topically (e.g. to the eye, to the skin or to the lung and/or airways) in the form, e.g., of eye-drops, creams, solutions, suspensions, heptafluoroalkane (HFA) aerosols and dry powder formulations; or systemically, e.g. by oral administration in the form of tablets, capsules, syrups, powders or granules; or by parenteral administration in the form of solutions or suspensions; or by subcutaneous administration; or by rectal administration in the form of suppositories; or transdermally. In a further embodiment, the pharmaceutical composition is in the form of a suspension, tablet, capsule, powder, granule or suppository.

In an embodiment of the invention, the active ingredient is administered orally. Oral administration may involve swallowing, so that the compound enters the gastrointestinal tract, and/or buccal, lingual, or sublingual administration by which the compound enters the blood stream directly from the mouth.

Formulations suitable for oral administration include solid plugs, solid microparticulates, semi-solid and liquid (including multiple phases or dispersed systems) such as tablets; soft or hard capsules containing multi- or nano-particulates, liquids, emulsions or powders; lozenges (including liquid-filled); chews; gels; fast dispersing dosage forms; films; ovules; sprays; and buccal/mucoadhesive patches.

Liquid (including multiple phases and dispersed systems) formulations include emulsions, suspensions, solutions, syrups and elixirs. Such formulations may be presented as fillers in soft or hard capsules. Liquid formulations may also be prepared by the reconstitution of a solid, for example, from a sachet.

The solid form of the invention may also be used in fast-dissolving, fast-disintegrating dosage forms such as those described in Liang and Chen, Expert Opinion in Therapeutic Patents, 2001, 11 (6), 981-986.

The formulation of tablets is discussed in Pharmaceutical Dosage Forms: Tablets, Vol. 1, by H. Lieberman and L. Lachman (Marcel Dekker, New York, 1980).

The invention will now be illustrated by the following non-limiting examples. In the examples the following figures are presented:

FIG. 1: X-ray powder diffraction pattern of Form 1 of the compound of Formula A (Example 1).

FIG. 2: STA of Form 1 of the compound of Formula A (Example 1).

FIG. 3: X-ray powder diffraction pattern of Form 2 of the hydrochloride salt of the compound of Formula A (Example 2).

FIG. 4: X-ray powder diffraction pattern of Form 3 of the sulfate salt of the compound of Formula A (Example 3).

### GENERAL EXPERIMENTAL DETAILS

In the following examples, the following abbreviations and definitions are used:

| | |
|---|---|
| aq | Aqueous solution |
| DCM | Dichloromethane |
| DMF | N,N-Dimethylformamide |
| DMSO | Dimethyl sulfoxide |
| DSC | Differential Scanning Calorimetry |
| EtOAc | Ethyl Acetate |
| HATU | 2-(3H-[1,2,3]triazolo[4,5-b]pyridin-3-yl)-1,1,3,3- |
| | tetramethylisouronium hexafluorophosphate(V) |
| hrs | Hours |
| HOBt | Hydroxybenzotriazole |
| IPA | 2-Propanol/Propan-2-ol/Iso-propanol |
| LCMS | Liquid chromatography mass spectrometry |
| Me | Methyl |
| MeCN | Acetonitrile |
| MeOH | Methanol |
| min | Minutes |
| MS | Mass spectrum |
| NMR | Nuclear magnetic resonance spectrum - NMR spectra |
| | were recorded at a frequency of 400 MHz unless |
| | otherwise indicated |
| Pet. Ether | Petroleum ether fraction boiling at 60-80° C. |
| Ph | Phenyl |
| STA | Simultaneous Thermal Analysis |
| SWFI | Sterile water for injection |
| rt | room temperature |
| THF | Tetrahydrofuran |
| TFA | Trifluoroacetic acid |
| XRPD | X-ray powder diffraction |

All reactions were carried out under an atmosphere of nitrogen unless specified otherwise.

¹H NMR spectra were recorded on a Bruker (400 MHz) or on a JEOL (400 MHz) spectrometer with reference to deuterium solvent and at rt.

Molecular ions were obtained using LCMS which was carried out using a Chromolith Speedrod RP-18e column, 50×4.6 mm, with a linear gradient 10% to 90% 0.1% HCO₂H/MeCN into 0.1% HCO₂H/H₂O over 13 min, flow rate 1.5 mL/min, or using Agilent, X-Select, acidic, 5-95% MeCN/water over 4 min. Data was collected using a Thermofinnigan Surveyor MSQ mass spectrometer with electospray ionisation in conjunction with a Thermofinnigan Surveyor LC system.

Alternatively, molecular ions were obtained using LCMS which was carried out using an Agilent Poroshell 120 EC-C18 (2.7 μm, 3.0×50 mm) column with 0.1% v/v Formic acid in water [eluent A]; MeCN [eluent B]; Flow rate 0.8 mL/min and 1.5 minutes equilibration time between samples, gradient shown below. Mass detection was afforded with API 2000 mass spectrometer (electrospray).

Gradient:

| Time (min) | Eluent A (%) | Eluent B (%) |
|---|---|---|
| 0.00 | 95 | 5 |
| 0.20 | 95 | 5 |
| 2.00 | 5 | 95 |
| 3.00 | 5 | 95 |
| 3.25 | 95 | 5 |
| 3.50 | 95 | 5 |

Where products were purified by flash chromatography, ‘silica’ refers to silica gel for chromatography, 0.035 to 0.070 mm (220 to 440 mesh) (e.g. Merck silica gel 60), and an applied pressure of nitrogen up to 10 p.s.i accelerated column elution. Reverse phase preparative HPLC purifications were carried out using a Waters 2525 binary gradient pumping system at flow rates of typically 20 mL/min using a Waters 2996 photodiode array detector.

All solvents and commercial reagents were used as received.

Chemical names were generated using automated software such as the Autonom software provided as part of the ISIS Draw package from MDL Information Systems or the Chemaxon software provided as a component of MarvinSketch or as a component of the IDBS E-WorkBook.

X-Ray Powder Diffraction patterns were collected on a Philips X-Pert MPD diffractometer and analysed using the following experimental conditions:

Scan parameters:

| | |
|---|---|
| Scan Axis: | Gonio |
| Start Position [°2θ]: | 4.0084 |
| End Position [°2θ]: | 39.9804 |
| Step Size [°2θ]: | 0.0170 |
| Scan Step Time [s]: | 10.1600 |
| Scan Type: | Continuous |
| PSD Mode: | Scanning |
| PSD Length [°2θ]: | 2.12 |
| Offset [°2θ]: | 0.0000 |
| Divergence Slit Type: | Automatic |
| Irradiated Length [mm] | 10.00 |
| Specimen Length [mm]: | 10.00 |
| Measurement Temperature [° C.]: | 25.00 |
| Anode Material: | Cu |
| K-Alpha1 [Å]: | 1.54060 |
| K-Alpha2 [Å]: | 1.54443 |
| K-A2/K-A1 Ratio: | 0.50000 |
| Generator Settings: | 40 mA, 40 kV |
| Diffractometer Type: | 0000000011038600 |
| Diffractometer Number: | 0 |
| Goniometer Radius [mm]: | 240.00 |
| Dist. Focus-Diverg. Slit [mm]: | 100.00 |
| Incident Beam Monochromator: | No |
| Spinning: | Yes |

Approximately 5 mg of sample under analysis was gently compressed on the XRPD zero back ground single obliquely cut silica sample holder. The sample was then loaded into the diffractometer for analysis.

Simultaneous Thermal Analysis (STA) data were collected using the following method: Approximately 5 mg of sample was accurately weighed into a ceramic crucible and it was placed into the chamber of Perkin-Elmer STA 600 TGA/DTA analyzer at ambient temperature. The sample was then heated at a rate of 10° C./min, typically from 25° C. to 300° C., during which time the change in weight was monitored as well as DTA signal. The purge gas used was nitrogen at a flow rate of 20 cm³/min.

### Synthetic Examples

### Example 1—Form 1 of N-[(3-fluoro-4-methoxypyridin-2-yl)methyl]-1-({4-[(2-oxopyridin-1-yl)methyl]phenyl}methyl)-3-(trifluoromethyl)pyrazole-4-carboxamide

### 3-Fluoro-4-methoxy-pyridine-2-carbonitrile

To a large microwave vial, cyanocopper (1.304 g, 14.6 mmol) was added to a solution of 2-bromo-3-fluoro-4-methoxypyridine (1 g, 4.9 mmol) in DMF (5 mL). The reaction vial was sealed and heated to 100° C. for 16 hrs. The reaction mixture was diluted with water (20 mL) and EtOAc (20 mL). The thick suspension was sonicated and required additional water (40 mL) and EtOAc (2×50 mL) with sonication to break-up the solid precipitated. The combined layers were filtered through a plug of Celite and the organic layer isolated, washed with brine (50 mL), dried over MgSO₄, filtered and the solvent removed under reduced pressure to give a pale green solid identified as 3-fluoro-4-methoxy-pyridine-2-carbonitrile (100 mg, 0.58 mmol, 12% yield)

### (3-Fluoro-4-methoxy-pyridin-2-ylmethyl)-carbamic acid tert-butyl ester

3-Fluoro-4-methoxy-pyridine-2-carbonitrile (100 mg, 0.58 mmol) was dissolved in anhydrous MeOH (10 mL, 247 mmol) and nickel chloride hexahydrate (14 mg, 0.058 mmol) was added followed by di-tert-butyl dicarbonate (255 mg, 1.16 mmol). The resulting pale green solution was cooled in an ice-salt bath to −5° C. and then sodium borohydride (153 mg, 4.1 mmol) was added portionwise maintaining the reaction temperature ˜0° C. The deep brown solution was left to stir at 0° C. and slowly allowed to warm to rt and then left to stir at rt for 3 hrs. The reaction mixture was evaporated to dryness at 40° C. to afford a black residue which was diluted with DCM (10 mL) and washed with sodium hydrogen carbonate (aq) (10 mL). An emulsion formed so the organics were separated via a phase separating cartridge and concentrated. The crude liquid was purified by chromatography eluting with EtOAc/iso-hexane to afford (3-fluoro-4-methoxy-pyridin-2-ylmethyl)-carbamic acid tert-butyl ester as a clear yellow oil (108 mg, 62% yield)

[MH]⁺=257

### (3-Fluoro-4-methoxy-pyridin-2-yl)-methylamine dihydrochloride salt

(3-Fluoro-4-methoxy-pyridin-2-ylmethyl)-carbamic acid tert-butyl ester (108 mg, 0.36 mmol) was taken up in iso-propyl alcohol (1 mL) and then HCl (6 N in iso-propyl alcohol) (1 mL, 0.58 mmol) was added at rt and left to stir at 40° C. for 2 hrs. The reaction mixture was concentrated under reduced pressure and then triturated with diethyl ether and sonicated to give a cream coloured solid (75 mg, 55% yield) identified as (3-fluoro-4-methoxy-pyridin-2-yl)-methylamine dihydrochloride salt.

[MH]⁺=157

### 1-(4-Chloromethyl-benzyl)-3-trifluoromethyl-1H-pyrazole-4-carboxylic acid ethyl ester

Polymer-supported triphenylphospine (3.0 mmol/g, 1.0 g) was swollen in THF/DCM (1:1, 100 mL). Under a nitrogen atmosphere ethyl 3-trifluoromethyl-1H-pyrazole-4-carboxylate (1.0 g, 4.8 mmol) and 4-(chloromethyl)benzylalcohol (903 mg, 5.8 mmol) were added followed by a solution of diisopropyl azodicarboxylate (1.46 g, 7.2 mmol) in THF/DCM (1:1, 10 mL) over a period of 30 min. The reaction mixture was stirred at rt for 18 hrs, the mixture was filtered and the resin was washed with 3 cycles of DCM/MeOH (15 mL). The combined filtrates were evaporated in vacuo. Two main products were identified which were separated by flash chromatography (silica), eluent 20% EtOAc, 80% Pet Ether, to give colourless white solids. The second product that eluted was identified as the title compound (790 mg, 47%).

[MH]⁺=347.1

### 1-[4-(2-Oxo-2H-pyridin-1-ylmethyl)-benzyl]-3-trifluoromethyl-1H-pyrazole-4-carboxylic acid ethyl ester

1-(4-Chloromethyl-benzyl)-3-trifluoromethyl-1H-pyrazole-4-carboxylic acid ethyl ester (790 mg, 2.3 mmol) was dissolved in acetone (150 mL). 2-Hydroxypyridine (260 mg, 2.7 mmol) and K₂CO₃ (945 mg, 6.8 mmol) were added and the reaction mixture was stirred at 50° C. for 3 hrs after which time the solvent was removed in vacuo. The residue was taken up in EtOAc (100 mL) and washed with water (1×30 mL), brine (1×30 mL), dried (Na₂SO₄), and evaporated in vacuo. The residue was purified by flash chromatography (silica), eluent 3% MeOH, 97% CHCl₃, to give a colourless oil identified as the title compound (670 mg, 1.7 mmol).

[M+H]⁺=406.2

### 1-[4-(2-Oxo-2H-pyridin-1-ylmethyl)-benzyl]-3-trifluoromethyl-1H-pyrazole-4-carboxylic acid

1-[4-(2-Oxo-2H-pyridin-1-ylmethyl)-benzyl]-3-trifluoromethyl-1H-pyrazole-4-carboxylic acid ethyl ester (670 mg, 1.7 mmol) was dissolved in THF (50 mL) and water (5 mL) and lithium hydroxide (19 8 mg, 8.3 mmol) was added. The reaction mixture was stirred at 50° C. for 18 hrs after which time the solvent was concentrated in vacuo and the residue taken up in EtOAc (50 mL). The aqueous layer was separated and acidified with 1 M HCl to pH 2 and extracted with CHCl₃ (3×50 mL). The combined organic extracts were washed with water (1×30 mL), brine (1×30 mL), dried (Na₂SO₄), filtered and evaporated in vacuo to give a white solid identified as the title compound (580 mg, 1.5 mmol, 93%).

[M+H]⁺=378.2

### N-[(3-Fluoro-4-methoxypyridin-2-yl)methyl]-1-({4-[(2-oxopyridin-1-yl)methyl]phenyl}methyl)-3-(trifluoromethyl)pyrazole-4-carboxamide

1-[4-(2-Oxo-2H-pyridin-1-ylmethyl)-benzyl]-3-trifluoromethyl-1H-pyrazole-4-carboxylic acid (150 mg, 0.4 mmol) was dissolved in DCM (30 mL). N,N,N′,N′-tetramethyl-O-(1H-benzotriazol-1-yl)uronium hexafluorophosphate (181 mg, 0.48 mmol) and N,N-diisopropylethyamine (77 mg, 0.6 mmol) were added at rt. After 20 min (3-fluoro-4-methoxy-pyridin-2-yl)methylamine (68 mg, 0.44 mmol) was added and the reaction mixture stirred at rt for 18 hrs. The reaction mixture was diluted with CHCl₃ (50 mL), this solution was washed with saturated NaHCO₃ (aq) (1×30 mL), water (1×30 mL), brine (1×30 mL), dried (Na₂SO₄), and evaporated in vacuo to give a yellow oil. The residue was purified by flash chromatography (silica), eluent 3% MeOH, 97% CHCl₃ and reduced in vacuo to afford a white solid identified as Form 1 of the title compound (116 mg, 0.23 mmol, 57%).

[M+H]⁺=516.3

¹H NMR (d6-DMSO, 400 MHz) δ3.92 (3H, s), 4.49 (2H, dd, J=5.6, 2.0 Hz), 5.08 (2H, s), 5.40 (2H, s), 6.21-6.24 (1H, m), 6.40 (1H, d, J=9.0 Hz), 7.16-7.25 (1H, m), 7.29 (4H, s), 7.39-7.43 (1H, m), 7.76 (1H, dd, J=6.8, 2.0 Hz), 8.21 (1H, d, J=5.5 Hz), 8.44 (1H, s), 8.70 (1H, t, J=5.4 Hz) ppm.

An XRPD diffractogram of Form 1 is shown in FIG. 1.

Peak position table:

| No. | Pos. [°2θ] | Height [cts] | Rel. Int. [%] |
|---|---|---|---|
| 1 | 6.7 | 67.7 | 10.9 |
| 2 | 8.2 | 57.3 | 9.2 |
| 3 | 9.5 | 112.9 | 18.2 |
| 4 | 11.0 | 74.5 | 12.0 |
| 5 | 11.6 | 29.5 | 4.8 |
| 6 | 13.3 | 308.9 | 49.7 |
| 7 | 13.7 | 114.4 | 18.4 |
| 8 | 14.5 | 94.8 | 15.3 |
| 9 | 15.6 | 151.8 | 24.4 |
| 10 | 17.3 | 621.2 | 100.0 |
| 11 | 18.8 | 98.4 | 15.8 |
| 12 | 19.1 | 205.9 | 33.1 |
| 13 | 19.7 | 65.4 | 10.5 |
| 14 | 20.0 | 64.9 | 10.5 |
| 15 | 20.5 | 218.4 | 35.2 |
| 16 | 21.3 | 89.2 | 14.4 |
| 17 | 22.1 | 101.5 | 16.4 |
| 18 | 22.9 | 261.3 | 42.1 |
| 19 | 23.9 | 219.7 | 35.4 |
| 20 | 24.3 | 262.8 | 42.3 |
| 21 | 24.5 | 281.2 | 45.3 |
| 22 | 25.2 | 129.1 | 20.8 |
| 23 | 25.7 | 126.8 | 20.4 |
| 24 | 26.2 | 79.3 | 12.8 |
| 25 | 27.7 | 115.0 | 18.5 |
| 26 | 28.2 | 92.2 | 14.8 |
| 27 | 29.1 | 85.0 | 13.7 |
| 28 | 29.6 | 58.6 | 9.4 |
| 29 | 31.5 | 73.9 | 11.9 |
| 30 | 33.1 | 15.4 | 2.5 |
| 31 | 34.0 | 17.0 | 2.7 |
| 32 | 34.8 | 28.9 | 4.7 |
| 33 | 36.4 | 47.6 | 7.7 |
| 34 | 38.2 | 12.9 | 2.1 |

### Simultaneous Thermal Analysis (STA)

The STA data for Form 1 are shown in FIG. 2.

### Example 2—Form 2 of N-[(3-fluoro-4-methoxypyridin-2-yl)methyl]-1-({4-[(2-oxopyridin-1-yl)methyl]phenyl}methyl)-3-(trifluoromethyl)pyrazole-4-carboxamide hydrochloride

To a suspension of N-[(3-fluoro-4-methoxypyridin-2-yl)methyl]-1-({4-[(2-oxopyridin-1-yl)methyl]phenyl}methyl)-3-(trifluoromethyl)pyrazole-4-carboxamide (10 mg) in acetonitrile (100 μL) was added 1.1 equivalents of 5 M hydrochloric acid (aq) (4.3 μL). The mixture was mixed well and temperature cycled between ambient and 40° C. for 18-24 hrs. The solvent was evaporated under nitrogen and the residue dried in vacuo for 24 hrs at 40° C. to afford Form 2 of N-[(3-fluoro-4-methoxypyridin-2-yl)methyl]-1-({4-[(2-oxopyridin-1-yl)methyl]phenyl}methyl)-3-(trifluoromethyl)pyrazole-4-carboxamide hydrochloride.

An XRPD diffractogram of Form 2 is shown in FIG. 3.

Peak position table:

| No. | Pos. [°2θ] | Height [cts] | Rel. Int. [%] |
|---|---|---|---|
| 1 | 4.3 | 43.4 | 15.7 |
| 2 | 5.4 | 21.8 | 7.9 |
| 3 | 7.3 | 201.8 | 72.8 |
| 4 | 7.7 | 60.1 | 21.7 |
| 5 | 7.8 | 64.4 | 23.2 |
| 6 | 8.6 | 179.3 | 64.7 |
| 7 | 9.2 | 25.2 | 9.1 |
| 8 | 9.9 | 38.8 | 14.0 |
| 9 | 11.2 | 138.4 | 49.9 |
| 10 | 11.6 | 178.6 | 64.4 |
| 11 | 12.3 | 7.9 | 2.9 |
| 12 | 13.5 | 121.7 | 43.9 |
| 13 | 13.9 | 63.5 | 22.9 |
| 14 | 14.3 | 172.2 | 62.1 |
| 15 | 14.7 | 113.7 | 41.0 |
| 16 | 15.3 | 20.0 | 7.2 |
| 17 | 16.2 | 200.1 | 72.2 |
| 18 | 17.2 | 176.8 | 63.8 |
| 19 | 17.7 | 218.0 | 78.6 |
| 20 | 18.3 | 277.3 | 100.0 |
| 21 | 18.8 | 130.8 | 47.2 |
| 22 | 19.8 | 151.3 | 54.6 |
| 23 | 20.0 | 110.9 | 40.0 |
| 24 | 20.3 | 55.3 | 20.0 |
| 25 | 20.9 | 80.2 | 28.9 |
| 26 | 21.4 | 49.5 | 17.9 |
| 27 | 21.7 | 171.5 | 61.8 |
| 28 | 22.1 | 74.4 | 26.8 |
| 29 | 22.7 | 124.7 | 45.0 |
| 30 | 23.1 | 33.3 | 12.0 |
| 31 | 23.5 | 48.9 | 17.6 |
| 32 | 24.2 | 99.1 | 35.8 |
| 33 | 24.7 | 176.3 | 63.6 |
| 34 | 25.3 | 109.4 | 39.5 |
| 35 | 26.1 | 177.4 | 64.0 |
| 36 | 26.6 | 122.4 | 44.2 |
| 37 | 26.9 | 100.4 | 36.2 |
| 38 | 28.0 | 156.0 | 56.3 |
| 39 | 29.5 | 93.1 | 33.6 |
| 40 | 29.8 | 76.8 | 27.7 |
| 41 | 30.5 | 100.9 | 36.4 |
| 42 | 31.4 | 49.9 | 18.0 |
| 43 | 31.8 | 31.4 | 11.3 |
| 44 | 32.3 | 24.2 | 8.7 |
| 45 | 32.8 | 16.3 | 5.9 |
| 46 | 34.2 | 52.3 | 18.9 |
| 47 | 34.9 | 52.8 | 19.0 |
| 48 | 35.9 | 92.0 | 33.2 |
| 49 | 37.1 | 26.0 | 9.4 |
| 50 | 37.9 | 17.8 | 6.4 |

### Example 3—Form 3 of N-[(3-fluoro-4-methoxypyridin-2-yl)methyl]-1-({4-[(2-oxopyridin-1-yl)methyl]phenyl}methyl)-3-(trifluoromethyl)pyrazole-4-carboxamide sulfate

To a suspension of N-[(3-fluoro-4-methoxypyridin-2-yl)methyl]-1-({4-[(2-oxopyridin-1-yl)methyl]phenyl}methyl)-3-(trifluoromethyl)pyrazole-4-carboxamide (7 mg) in acetonitrile (100 μL) was added 1.1 equivalents of 5 M sulfuric acid (aq) (4.3 μL). The mixture was mixed well and temperature cycled between ambient and 40° C. for 18-24 hrs. The solvent was evaporated under nitrogen and the residue dried in vacuo for 24 hrs at 40° C. to afford Form 3 of N-[(3-fluoro-4-methoxypyridin-2-yl)methyl]-1-({4-[(2-oxopyridin-1-yl)methyl]phenyl}methyl)-3-(trifluoromethyl)pyrazole-4-carboxamide sulfate.

An XRPD diffractogram of Form 3 is shown in FIG. 4.

Peak position table:

| No. | Pos. [°2θ] | Height [cts] | Rel. Int. [%] |
|---|---|---|---|
| 1 | 4.7 | 84.1 | 37.8 |
| 2 | 6.4 | 106.5 | 47.8 |
| 3 | 8.5 | 14.6 | 6.5 |
| 4 | 9.1 | 73.9 | 33.2 |
| 5 | 10.9 | 12.9 | 5.8 |
| 6 | 12.8 | 40.4 | 18.1 |
| 7 | 13.9 | 38.5 | 17.3 |
| 8 | 15.1 | 222.7 | 100.0 |
| 9 | 15.9 | 32.1 | 14.4 |
| 10 | 16.4 | 132.8 | 59.6 |
| 11 | 17.9 | 37.6 | 16.9 |
| 12 | 18.4 | 125.5 | 56.4 |
| 13 | 18.8 | 123.3 | 55.3 |
| 14 | 19.5 | 165.9 | 74.5 |
| 15 | 20.1 | 32.5 | 14.6 |
| 16 | 20.6 | 47.1 | 21.2 |
| 17 | 21.8 | 78.1 | 35.1 |
| 18 | 22.5 | 53.5 | 24.0 |
| 19 | 22.8 | 90.1 | 40.4 |
| 20 | 23.5 | 109.7 | 49.3 |
| 21 | 23.9 | 84.6 | 38.0 |
| 22 | 24.2 | 136.9 | 61.5 |
| 23 | 25.2 | 190.1 | 85.4 |
| 24 | 25.5 | 176.0 | 79.0 |
| 25 | 27.3 | 74.4 | 33.4 |
| 26 | 28.3 | 37.4 | 16.8 |
| 27 | 29.0 | 13.8 | 6.2 |
| 28 | 30.3 | 43.9 | 19.7 |
| 29 | 38.3 | 11.0 | 5.0 |

### Biological Methods

The ability of the compound of formula A to inhibit plasma kallikrein may be determined using the following biological assays. Data for a reference compound, Example 41 of WO2016/083820 (N-[(3-fluoro-4-methoxypyridin-2-yl)methyl]-3-(methoxymethyl)-1-({4-[(2-oxopyridin-1-yl)methyl]phenyl}methyl)pyrazole-4-carboxamide) is also provided for comparative purposes.

### Determination of the IC₅₀ for Plasma Kallikrein

Plasma kallikrein inhibitory activity in vitro was determined using standard published methods (see e.g. Johansen et al., Int. J. Tiss. Reac. 1986, 8, 185; Shori et al., Biochem. Pharmacol., 1992, 43, 1209; Stürzebecher et al., Biol. Chem. Hoppe-Seyler, 1992, 373, 1025). Human plasma kallikrein (Protogen) was incubated at 25° C. with the fluorogenic substrate H-DPro-Phe-Arg-AFC and various concentrations of the test compound. Residual enzyme activity (initial rate of reaction) was determined by measuring the change in optical absorbance at 410 nm and the IC₅₀ value for the test compound was determined.

Data acquired from this assay are shown in Table 1.

Compounds were further screened for inhibitory activity against the related enzyme KLK1. The ability of the compounds to inhibit KLK1 may be determined using the following biological assay:

### Determination of the IC₅₀ for KLK1

KLK1 inhibitory activity in vitro was determined using standard published methods (see e.g. Johansen et al., Int. J. Tiss. Reac. 1986, 8, 185; Shori et al., Biochem. Pharmacol., 1992, 43, 1209; Stürzebecher et al., Biol. Chem. Hoppe-Seyler, 1992, 373, 1025). Human KLK1 (Callbiochem) was incubated at 25° C. with the fluorogenic substrate H-DVal-Leu-Arg-AFC and various concentrations of the test compound. Residual enzyme activity (initial rate of reaction) was determined by measuring the change in optical absorbance at 410 nm and the IC₅₀ value for the test compound was determined.

Data acquired from this assay are shown in Table 1.

Compounds were also screened for inhibitory activity against the related enzyme FXIa. The ability of the compounds to inhibit FXIa may be determined using the following biological assay:

### Determination of the % Inhibition for FXIa

FXIa inhibitory activity in vitro was determined using standard published methods (see e.g. Johansen et al., Int. J. Tiss. Reac. 1986, 8, 185; Shori et al., Biochem. Pharmacol., 1992, 43, 1209; Stürzebecher et al., Biol. Chem. Hoppe-Seyler, 1992, 373, 1025). Human FXIa (Enzyme Research Laboratories) was incubated at 25° C. with the fluorogenic substrate Z-Gly-Pro-Arg-AFC and 40 μM of the test compound (or alternatively at various concentrations of the test compound in order to determine IC₅₀). Residual enzyme activity (initial rate of reaction) was determined by measuring the change in optical absorbance at 410 nm and the IC₅₀ value for the test compound was determined.

Data acquired from this assay are shown in Table 1.

Compounds were also screened for inhibitory activity against the related enzyme FXIIa. The ability of the compounds to inhibit FXIIa may be determined using the following biological assay:

### Determination of the IC₅₀ for FXIIa

Factor XIIa inhibitory activity in vitro was determined using standard published methods (see e.g. Shori et al., Biochem. Pharmacol., 1992, 43, 1209; Baeriswyl et al., ACS Chem. Biol., 2015, 10 (8) 1861; Bouckaert et al., European Journal of Medicinal Chemistry 110 (2016) 181). Human Factor XIIa (Enzyme Research Laboratories) was incubated at 25° C. with the fluorogenic substrate H-DPro-Phe-Arg-AFC and various concentrations of the test compound. Residual enzyme activity (initial rate of reaction) was determined by measuring the change in optical absorbance at 410 nm and the IC₅₀ value for the test compound was determined.

Data acquired from this assay are shown in Table 1.

TABLE 1

| | IC₅₀ | IC₅₀ | IC₅₀ | % Inhibition | IC₅₀ |
|---|---|---|---|---|---|
| | (human | (human | (human | @ 40 μM | (human |
| Compound | PKal) nM | KLK1) nM | FXIa) nM | (human FXIa) | FXIIa) nM |
| Example 41 of | 3.3 | >40,000 | >40,000 | 0 | >40,000 |
| WO2016/083820 | | | | | |
| Compound of Formula A | 3.2 | >4000 | >40,000 | 2 | >4,000 |

### Determination of Enzyme Selectivity

Human serine protease enzymes plasmin, thrombin and trypsin were assayed for enzymatic activity using an appropriate fluorogenic substrate. Protease activity was measured by monitoring the accumulation of liberated fluorescence from the substrate over 5 minutes. The linear rate of fluorescence increase per minute was expressed as percentage (%) activity. The Km for the cleavage of each substrate was determined by standard transformation of the Michaelis-Menten equation. The compound inhibitor assays were performed at substrate Km concentration and activities were calculated as the concentration of inhibitor giving 50% inhibition (IC₅₀ ) of the uninhibited enzyme activity (100%).

Data acquired from these assays are shown in Table 2 below:

TABLE 2
Selectivity data

| | IC₅₀ (nM) | | |
|---|---|---|---|
| Compound | Plasmin | Thrombin | Trypsin |
| Compound of | >40000 | >40000 | >40000 |
| Formula A | | | |

### In Vitro ADME Data

In vitro permeability was determined using the Caco-2 model for oral absorption. The methodology was adapted from standard published methods (Wang Z, Hop C. E., Leung K. H. and Pang J. (2000) J Mass Spectrom 35(1); 71-76). The Caco-2 monolayers were established in a Biocoat™ HTS fibrillar collagen 24 well multiwell insert system (1.0 μm, PET membrane, Corning 354803) in which 200,000 cells were seeded into each insert and maintained over 3 days before being utilised in the permeability assay. For the assay, 50 μM test compound is added to the apical side of the inserts and incubated for 1 hour at 37° C. on a shaking platform (120 rpm). Apical to basolateral transport was determined by measuring the test article in both compartments by LCMS following the 1 hour incubation. The integrity of the Caco-2 monolayers was confirmed by two methods, (i) comparison of pre- and post-experiment transepithelial electrical resistance (TEER) and, (ii) assessment of Lucifer Yellow flux. The results are shown in Table 3 below:

TABLE 3
Permeability data

| | Caco-2 |
|---|---|
| Compound | (Papp ×10⁻⁶ cm/s) |
| Example 41 of WO2016/083820 | 9 |
| Compound of Formula A | 17 |

### Pharmacokinetics

Pharmacokinetic studies of the compounds in Table 4 were performed to assess the pharmacokinetics following a single oral dose in male Sprague-Dawley rats. Two rats were given a single po dose of 5 mL/kg of a nominal 2 mg/mL (10 mg/kg) composition of test compound in vehicle. Following dosing, blood samples were collected over a period of 24 hours. Sample times were 5, 15 and 30 minutes then 1, 2, 4, 6, 8 and 12 hours. Following collection, blood samples were centrifuged and the plasma fraction analysed for concentration of test compound by LCMS. Oral exposure data acquired from these studies are shown in Table 4 below:

TABLE 4
Oral exposure data

| | | Dose po | Cmax | Tmax |
|---|---|---|---|---|
| Compound | Vehicle | (mg/kg) | (ng/mL) | (min) |
| Example 41 of | 10% DMSO/10% | 10.5 | 1534 | 180 |
| WO2016/083820 | cremophor/80% SWFI | | | |
| Compound of | 10% DMSO/10% | 2.2 | 1802 | 30 |
| Formula A | cremophor/80% SWFI | | | |

1. A solid form of the compound of Formula A, 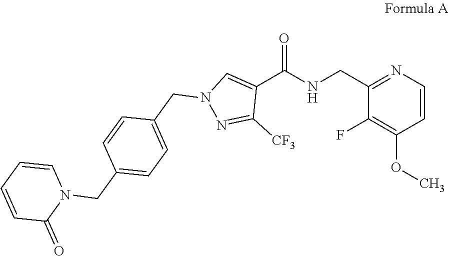 [SMILES: COc1ccnc(CNC(=O)c2cn(Cc3ccc(Cn4ccccc4=O)cc3)nc2C(F)(F)F)c1F]
which exhibits:
at least the following characteristic X-ray powder diffraction peaks (Cu Kα radiation, expressed in degrees 2θ) at approximately 6.7, 9.5, 11.0, 13.3 and 17.3;
an endothermic peak in its STA thermograph at 164±3° C.; or
combinations thereof.

2. The solid form according to claim 1 having an X-ray powder diffraction pattern substantially the same as that shown in FIG. 1.

3. The solid form according to claim 1, which exhibits an endothermic peak in its STA thermograph at 164±3° C.

4. The solid form according to claim 1 having an STA thermograph substantially the same as that shown in FIG. 2.

5. (canceled)

6. (canceled)

7. A solid form of the hydrochloride salt of the compound of Formula A, 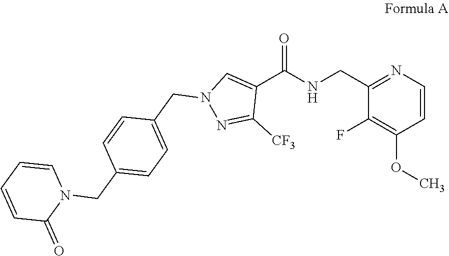 [SMILES: COc1ccnc(CNC(=O)c2cn(Cc3ccc(Cn4ccccc4=O)cc3)nc2C(F)(F)F)c1F]
which:
exhibits at least the following characteristic X-ray powder diffraction peaks (Cu Kα radiation, expressed in degrees 2θ) at approximately 7.3, 8.6, 11.6, 14.3, and 16.2; or
has an X-ray powder diffraction pattern substantially the same as that shown in FIG. 3; or a combination thereof.

8. (canceled)

9. (canceled)

10. A solid form of the sulfate salt of the compound of Formula A, 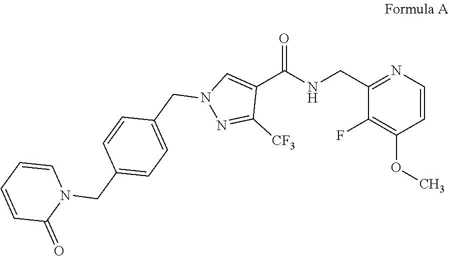 [SMILES: COc1ccnc(CNC(=O)c2cn(Cc3ccc(Cn4ccccc4=O)cc3)nc2C(F)(F)F)c1F]
which:
exhibits at least the following characteristic X-ray powder diffraction peaks (Cu Kα radiation, expressed in degrees 2θ) at approximately 4.7, 6.4, 9.1, 15.1, and 16.4; or
has an X-ray powder diffraction pattern substantially the same as that shown in FIG. 4; or a combination thereof.

11. (canceled)

12. (canceled)

13. A pharmaceutical composition comprising a solid form of claim 1, and a pharmaceutically acceptable adjuvant, diluent and/or carrier.

14. (canceled)

15. (canceled)

16. A method of treating a disease or condition mediated by plasma kallikrein, said method comprising administering to a mammal in need of such treatment a therapeutically effective amount of a solid form of claim 1.

17. (canceled)

18. The method of claim 16, wherein the disease or condition mediated by plasma kallikrein is impaired visual acuity, diabetic retinopathy, retinal vascular permeability associated with diabetic retinopathy, diabetic macular edema, hereditary angioedema, retinal vein occlusion, diabetes, pancreatitis, cerebral haemorrhage, nephropathy, cardiomyopathy, neuropathy, inflammatory bowel disease, arthritis, inflammation, septic shock, hypotension, cancer, adult respiratory distress syndrome, disseminated intravascular coagulation, blood coagulation during cardiopulmonary bypass surgery, or bleeding from post-operative surgery.

19. The method of claim 16, wherein the disease or condition mediated by plasma kallikrein is retinal vascular permeability associated with diabetic retinopathy, diabetic macular edema, or hereditary angioedema.

20. The method of claim 16, wherein the disease or condition mediated by plasma kallikrein is retinal vascular permeability associated with diabetic retinopathy, or diabetic macular edema.

21. The method of claim 16, wherein the disease or condition mediated by plasma kallikrein is hereditary angioedema.

22. The method of claim 16, wherein the disease or condition mediated by plasma kallikrein is diabetic macular edema.

23. The method according to claim 20, wherein said solid form is in a form suitable for injection into the ocular region of a mammal.

24. The method according to claim 23, wherein the solid form is in a form suitable for intra-vitreal injection.

25. A pharmaceutical composition comprising a solid form of claim 10, and a pharmaceutically acceptable adjuvant, diluent and/or carrier.

26. A method of treating a disease or condition mediated by plasma kallikrein, said method comprising administering to a mammal in need of such treatment a therapeutically effective amount of a solid form of claim 10.

27. The method of claim 26, wherein the disease or condition mediated by plasma kallikrein is impaired visual acuity, diabetic retinopathy, retinal vascular permeability associated with diabetic retinopathy, diabetic macular edema, hereditary angioedema, retinal vein occlusion, diabetes, pancreatitis, cerebral haemorrhage, nephropathy, cardiomyopathy, neuropathy, inflammatory bowel disease, arthritis, inflammation, septic shock, hypotension, cancer, adult respiratory distress syndrome, disseminated intravascular coagulation, blood coagulation during cardiopulmonary bypass surgery, or bleeding from post-operative surgery.

28. The method of claim 26, wherein the disease or condition mediated by plasma kallikrein is retinal vascular permeability associated with diabetic retinopathy, diabetic macular edema, or hereditary angioedema.

29. The method of claim 26, wherein the disease or condition mediated by plasma kallikrein is retinal vascular permeability associated with diabetic retinopathy, or diabetic macular edema.

30. The method of claim 26, wherein the disease or condition mediated by plasma kallikrein is hereditary angioedema.

31. The method of claim 26, wherein the disease or condition mediated by plasma kallikrein is diabetic macular edema.

32. The method according to claim 29, wherein said solid form is in a form suitable for injection into the ocular region of a mammal.

33. The method according to claim 32, wherein said solid form is in a form suitable for intra-vitreal injection.

34. A pharmaceutical composition comprising a solid form of claim 7, and a pharmaceutically acceptable adjuvant, diluent and/or carrier.

35. A method of treating a disease or condition mediated by plasma kallikrein, said method comprising administering to a mammal in need of such treatment a therapeutically effective amount of a solid form of claim 7.

36. The method of claim 35, wherein the disease or condition mediated by plasma kallikrein is impaired visual acuity, diabetic retinopathy, retinal vascular permeability associated with diabetic retinopathy, diabetic macular edema, hereditary angioedema, retinal vein occlusion, diabetes, pancreatitis, cerebral haemorrhage, nephropathy, cardiomyopathy, neuropathy, inflammatory bowel disease, arthritis, inflammation, septic shock, hypotension, cancer, adult respiratory distress syndrome, disseminated intravascular coagulation, blood coagulation during cardiopulmonary bypass surgery, or bleeding from post-operative surgery.

37. The method of claim 35, wherein the disease or condition mediated by plasma kallikrein is retinal vascular permeability associated with diabetic retinopathy, diabetic macular edema, or hereditary angioedema.

38. The method of claim 35, wherein the disease or condition mediated by plasma kallikrein is retinal vascular permeability associated with diabetic retinopathy, or diabetic macular edema.

39. The method of claim 35, wherein the disease or condition mediated by plasma kallikrein is hereditary angioedema.

40. The method of claim 35, wherein the disease or condition mediated by plasma kallikrein is diabetic macular edema.

41. The method according to claim 38, wherein said solid form is in a form suitable for injection into the ocular region of a mammal.

42. The method according to claim 41, wherein said solid form is in a form suitable for intra-vitreal injection.
